(19) United States
(12) Patent Application Publication
(10) Pub. No.: US 2004/0058993 A1
(43) Pub. Date: Mar. 25, 2004

(54) Inhibitor for the production of TNF alpha

(75) Inventors: Sonoko Ishizaki, Kawasaki-shi (JP); Ichiro Sonaka, Kawasaki-shi (JP); Yukio Iino, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC., Tokyo (JP)

(21) Appl. No.: 10/601,699

(22) Filed: Jun. 24, 2003

Related U.S. Application Data
(63) Continuation of application No. PCT/JP01/11112, filed on Dec. 18, 2001.

(30) Foreign Application Priority Data
Dec. 27, 2000 (JP) 2000-397522

(51) Int. Cl. A61K 31/255; A61K 31/185

(52) U.S. Cl. 514/517; 514/553

(57) ABSTRACT

The present invention provides a medicinal composition containing aminomethanesulfonic acid (including a salt or ester form) as an active ingredient. The inventive medicinal composition has an improved TNFα production inhibition as compared with glycine. The present invention also provides a method of treating liver diseases by administering the medicinal composition to a patient in need thereof. There are also provided a method for inhibiting the production of TNFα and, particularly, a method of treatment, amelioration and/or prevention of liver diseases utilizing the above method.

### CROSS REFERENCE TO RELATED CASES

The present application is a continuation of PCT/JP01/11112, filed on Dec. 18, 2001, which claims priority to Japanese Patent Application No. JP 2000-397522, filed on Dec. 27, 2000, which are hereby incorporated by reference in their entirety.

### BACKGROUND OF THE INVENTION

1. Field of the Invention

The present invention relates to a novel medicinal composition containing aminomethanesulfonic acid as an active ingredient. The present invention also relates to medicinal compositions containing, as an active ingredient, aminomethanesulfonic acid derivatives, such as salt, ester, etc., which can be converted to aminomethanesulfonic acid in a living body. The medicinal compositions of the present invention are useful as an inhibitor of TNFα production. These drugs can be used to treat liver diseases. The present invention also relates to a method for inhibiting the production of TNFα, particularly a method for treatment, amelioration and/or prevention of liver diseases and to a use of the above-mentioned active ingredient for a medicinal composition, preferably an inhibitor for the production of TNFα or a drug for liver diseases.

2. Discussion of the Background

Glycine, alanine and serine (especially glycine) have been proposed as an ingredient(s) for drugs or nutritive agents (nutritional preparation) for decreasing the tumor necrosis factor (TNF) level in patients where the level increases beyond the range of mediation of physiological homeostasis and local inflammation (refer to WO 96/25861 and JP-A-11-501301). However, the effect of these amino acids has not been sufficient. As such a critical need exists for development of drugs where the aforementioned effects are improved.

Accordingly, an object of the present invention is to provide a better inhibitor for the production of TNFα and to use it as drug(s) (drug for liver diseases, etc.) utilizing the action thereof.

### SUMMARY OF THE INVENTION

As a result of the extensive studies, the present inventors have carried out intensive investigations and, as a result, they have found that aminomethanesulfonic acid is excellent in an effectuating inhibiting the production of TNFα. On the basis of such a finding, the present invention has been achieved.

Thus, the present invention relates to a medicinal composition containing aminomethanesulfonic acid as an active ingredient. As will be mentioned later, the aminomethanesulfonic acid may be in a free form or in a form of various derivatives such as a salt or an ester.

When it is used as a drug (medicament), aminomethanesulfonic acid has an excellent TNFα production inhibiting activity; therefore, it can be used as an inhibitor for the production of TNFα. It is particularly appropriate as a drug for liver diseases.

The above active ingredient used for this invention is aminomethanesulfonic acid and it includes a form of derivative(s) which are able to be converted to aminomethanesulfonic acid in a living body, particularly in a living body of human being. Suitable derivatives include a salt of aminomethanesulfonic acid, an ester of aminomethanesulfonic acid, and the like. The ester may be represented by the following general formula (1).

H₂N—CH₂—SO₃R (1)

In the above formula, R may represent a substituent that constitutes the ester, the R being able to be converted to a hydrogen atom in a living body, particularly in a human body. With regard to such a substituent, an alkyl group having 1 to 6 carbons may be preferably exemplified.

The salt (a sulfonate) of aminomethanesulfonic acid may be a salt that can be converted to a free substance in a living body, particularly in a human body. Suitable examples include sodium salt, potassium salt, calcium salt, magnesium salt, ammonium salt (including a salt with an amine such as methylamine, dimethylamine, methylethylamine, triethylamine, etc.).

It is an essential condition for the medicinal composition of this invention to contain the above-mentioned active ingredient and, so far as the advantage of this invention that an inhibiting action for the production of TNFα is aimed is not disturbed. It is also possible to use other active ingredient(s) in either a mixed form or a combined form in addition to the inventive active ingredient in the drug. In the manufacture of a medicinal preparation for the medicinal composition in the present invention, it is further possible to appropriately select and use additional ingredients that are necessary for the manufacture of medicinal (pharmaceutical) preparations.

In another embodiment, the present invention relates to a method for inhibiting the production of TNFα by administering the aminomethanesulfonic acid to a living subject (body), and to a method for treatment, amelioration and/or prevention of liver diseases which is characterized in administering the aminomethanesulfonic acid to a living body (the aminomethanesulfonic acid may be in a form of salt or ester in both of the above-mentioned embodiments).

With regard to a mode for the administration, it is possible to adopt a thing that is in a form of the above-mentioned medicinal composition of the present invention.

In still another embodiment, the present invention relates to the use of aminomethanesulfonic acid as a medicinal composition, particularly an inhibitor for the production of TNFα, a drug for liver diseases, etc (where the aminomethanesulfonic acid may be in a form of salt or ester).

Such a medicinal composition for administration to a patient in need thereof is as same as that illustrated hereinabove.

The above objects highlight certain aspects of the present invention. Additional objects, aspects and embodiments of the present invention ate found in the following detailed description of the present invention.

### BRIEF DESCRIPTION OF THE FIGURES

A more complete appreciation of the present invention and many of the attendant advantages thereof will be readily obtained as the same becomes better understood by reference to the following Figures in conjunction with the detailed description below.

FIG. 1 diagrams the result where the amount of production of TNFαis measured in Example 1. AMS: aminomethanesulfonic acid; Gly: glycine.

FIG. 2 shows the result of measurement of ALT in plasma obtained in Example 2. AMS: aminomethanesulfonic acid; *p<0.05.

FIG. 3 shows the result of measurement of AST in plasma obtained in Example 2. AMS: aminomethanesulfonic acid; *p<0.05.

### DETAILED DESCRIPTION OF THE INVENTION

Unless specifically defined, all technical and scientific terms used herein have the same meaning as commonly understood by a skilled artisan in the pharmaceutical and medicinal fields.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention is based in part on the Inventor's surprising discovery that TNFα production can be inhibited by administering a composition containing aminomethanesulfonic acid to a living subject (body).

The medicinal composition (medicament composition) of the present invention is preferably used as a drug that inhibits the production of TNFα (i.e., is an inhibitor for the production of TNFα). The medicinal composition is suitable for prevention, amelioration and/or treatment of liver diseases such as alcoholic, viral or drug-induced hepatitis, hepatic fibrosis, cirrhosis and fulminant hepatitis, inflammatory intestinal diseases, pancreatitis, arthritis, arteriosclerosis, sepsis, ischemic reperfusion injury, etc. It is particularly useful for prevention, amelioration and/or treatment of liver diseases.

The aminomethanesulfonic acid that is an active ingredient used in this invention is a known compound and can be easily prepared although it is convenient to use by purchasing it from a market (that manufactured by Tokyo Kasei Kogyo K. K., etc.).

In the formation of ester or salt when the aminomethanesulfonic acid is used in a form of ester or salt, aimed ester or salt can be easily prepared by utilizing a method known in the art where a sulfonate (sulfonic acid ester) is prepared from a sulfonic acid or by utilizing a salt formation step by addition of alkali.

When the active ingredient is used, for example, as a drug for liver diseases, the form of the medicinal preparation in the medicinal composition is not particularly limited but may be either an oral preparation or a parenteral preparation (such as an injection preparation).

With respect to the dose of the active ingredient (granular preparation/oral preparation containing aminomethanesulfonic acid) for a patient suffering, for example, from liver diseases, although it depends upon symptom of the patient, dosage form, etc., there may be used preferably approximately 1 mg to 10 g per day. More preferably the dosage is approximately 10 mg to 5 g per day and, most preferably, approximately 100 mg to 1 g per day. When the dosage form is an injection preparation for intravenous administration, the dose of approximately from one-twentieth to one-half of the above active ingredient used for the above oral preparation is sufficient.

Although a dosage form containing at least the essential active ingredient according to the present invention (the above-mentioned aminomethanesulfonic acid) can be administered to patients, it is also possible to use a medicinal composition achieving an inhibitory effect for the production of TNFα where a medicinal ingredient which is other than the active ingredient is also contained therein or combined therewith.

In the manufacture of the medicinal preparation in the medicinal composition, various pharmacologically-acceptable substances (i.e., auxiliary agents, etc.) for medicinal preparations may be added to the inventive medicinal composition. Such substances for medicinal preparations may be appropriately selected depending upon the dosage form of the preparation. For example, these include fillers, diluents, additives, disintegrating agents, binders, coating agents, lubricants, gliding agents, lubricating agents, flavoring agents, sweeteners, solubilizing agents, spices, dyes, nutrients (such as vitamins), and other additives that are suitable for being contained in the preparation. Further specific examples of the substances for medicinal preparations include magnesium carbonate, titanium dioxide, saccharide(s) such as lactose, mannitol, etc., talc, milk protein, gelatin, starch, cellulose and derivatives thereof, animal and plant oil(s), polyethylene glycol and solvent(s) such as aseptic water (sterilized water) and mono- and polyhydric alcohol (e.g., glycerol).

The drug (medicinal composition) of present invention can be prepared in various medicinal preparation forms or various dosage forms for oral administration, intraperitoneal administration, percutaneous administration, inhalation administration, etc. In order to make the medicinal ingredient used in the present invention into such various medicinal preparation forms, methods which have been known or which will be developed in future may be appropriately adopted.

With regard to the various forms of medicinal preparations, there may be exemplified appropriate preparation forms in solid or in solution such as granule, powder, coated tablet, tablet, diluted powder (powder medicine), (micro)capsule, suppository, syrup, juice, suspension, emulsion, dropping agent, solution for injection, preparation for extending the release of the active ingredient, and the like.

It goes without saying that the drug of the present invention in the above-exemplified dosage forms contains at least the above-mentioned ingredient (aminomethanesulfonic acid) in an amount that is effective for achieving the aimed medicinal effect.

As mentioned hereinabove, as another embodiment(s), the present invention also relates to a method for inhibiting the production of TNFα. This method comprises administering aminomethanesulfonic acid to a living subject (body). Accordingly, the present invention also relates to a method for treatment, amelioration and/or prevention of liver diseases by administering aminomethanesulfonic acid to a living body (in both of the above cases, the aminomethanesulfonic acid may be in a form of salt, ester or the like). In still another embodiment, the present invention also relates to a use of aminomethanesulfonic acid for a medicinal composition, particularly an inhibitor for the production of TNFα, a drug for liver diseases, etc. (the aminomethanesulfonic acid may be in a form of salt, ester or the like).

With regard to the invention(s) for the above embodiments, they may be easily carried out on the basis of the above-mentioned illustration for the medicinal composition of this invention or of the Examples that will be mentioned later or may be carried out by referring to the already known art if necessary.

In accordance with the present invention, an inhibitor for the production of TNFα containing aminomethanesulfonic acid (which may be in a form of salt, ester, etc.) as an active ingredient is provided and is able to be used for drugs (a drug for liver diseases, etc.) utilizing its action of inhibiting the production of TNFα.

There are also provided a use of the above-mentioned active ingredient for drugs, a method for inhibiting the production of TNFα and, particularly, a method for treatment, amelioration and/or prevention of liver diseases utilizing such a method.

Accordingly, this invention is quite useful in industry, particularly in the fields of medical treatment, drugs, etc.

Having generally described this invention, a further understanding can be obtained by reference to certain specific examples, which are provided herein for purposes of illustration only, and are not intended to be limiting unless otherwise specified.

### EXAMPLES

### Example 1

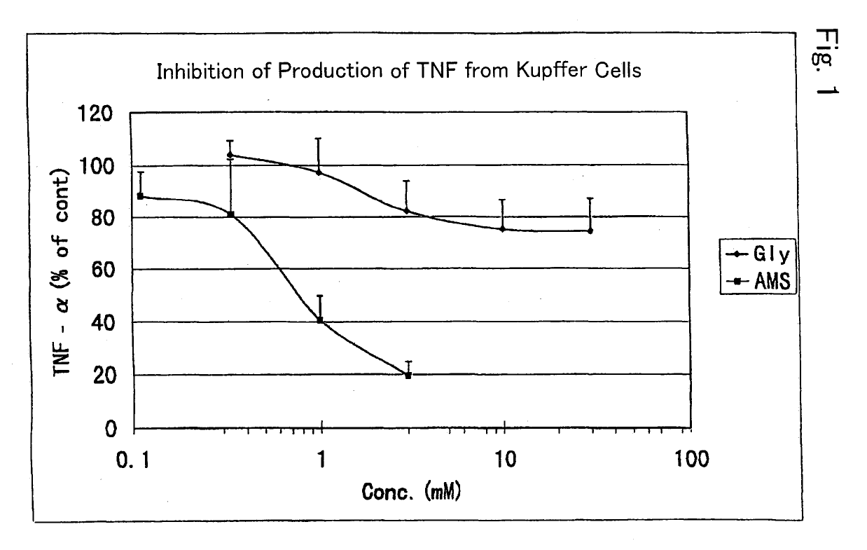

A male rat of SD strain (body weight: 200-300 g) was subjected to laparotomy under anesthetization with Nembutal, a collagenase solution was perfused from portal vein, then the liver was excised and Kupffer cells were separated by an elutriator method. The Kupffer cells were adjusted to 5×10⁵ cells/ml, each 100μ/well thereof were spread on a 96-well microplate and incubation was carried out for 48 hours using E-MEM (Eagle-MEM) and 10% FCS (fetal calf serum) to conduct the experiment. LPS (lipopoly saccharide) (10 μg/ml) and aminomethanesulofonic acid (AMS) or glycine were added at the same time thereto so as to make their concentrations 0.1 to 3 mM or 0.3 to 30 mM, respectively and the amount of TNFα produced in a supernatant liquid after incubation for 4 hours was measured by an ELISA (enzyme-linked immunosorbent assay). The result is shown in FIG. 1.

It is apparent from the result of FIG. 1 that aminomethanesulfonic acid inhibited the production of TNFα in a considerably strong manner as compared with glycine and its use in various kinds of drugs as an inhibitor for the production of TNFα can be expected.

### Example 2

Action of Aminomethanesulfonic Acid (AMS) to Necrosis of Hepatocytes (Method of Experiment)

A male rat of SD strain of 6 weeks age (150 g) was introduced, subjected to a preliminary breeding by giving CRF-1 (a feed manufactured by Oriental Yeast) and water for six days, fasted for one night and used for the experiment. During the fasting, a 10% aqueous solution of glucose was freely taken by the rat for preventing the lowering of blood-sugar level. D-galactosamine (600 mg/kg) adjusted to pH 6.8 was intraperitoneally administered using a 30% physiological saline solution. After 24 hours from administration of D-galactosamine, 700 μl of blood were collected from subclavian vein under anesthetization with ether and AST (aspartate aminotransferase) and ALT (alanine aminotransferase) in plasma were measured by a Fuji Dry Chem (an automated biochemical measuring apparatus for blood manufactured by Fuji Photo-Film). One hour before the administration of D-galactosamine, AMS was orally administered as a drug to be tested using a 0.3% aqueous solution of carboxymethyl cellulose (CMC).

(Administered Groups)

Group 1: 0.3% CMC n=8;

Group 2: AMS (1.0 g/kg)/0.3% CMC n=8.

(Result of Evaluation)

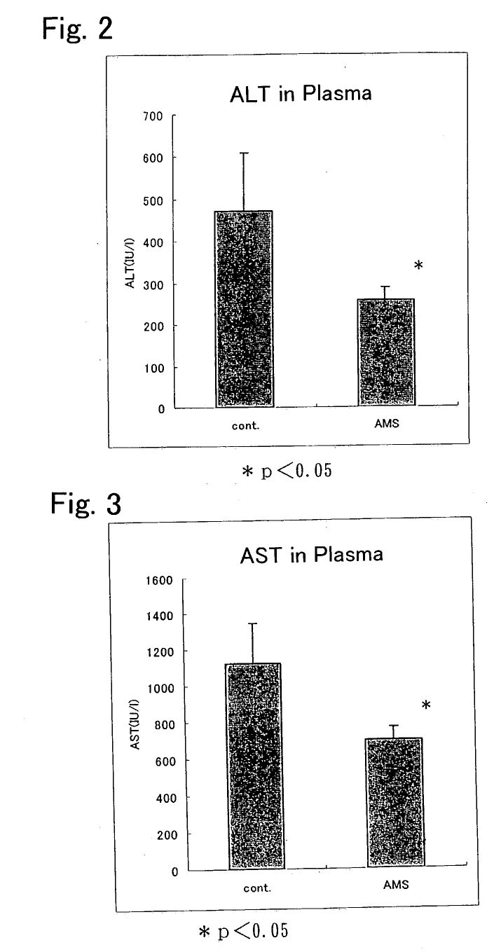

Results of the above measurement are shown in FIG. 2 and FIG. 3.

From these results, it was noted that, in the group administered with aminomethanesulfonic acid, a rise in AST and that in ALT in the plasma after 24 hours from administration of D-galactosamine were significantly inhibited.

Numerous modifications and variations on the present invention are possible in light of the above teachings. It is, therefore, to be understood that within the scope of the accompanying claims, the present invention may be practiced otherwise than as specifically described herein.

What we claim is:

1. A medicinal composition comprising aminomethanesulfonic acid as an active ingredient wherein the aminomethanesulfonic acid is in the free form or in a form of a salt or an ester.

2. The medicinal composition according to claim 1, wherein the aminomethanesulfonic acid is in a form of an ester.

3. The medicinal composition according to claim 2, wherein the ester is represented by the following formula (1)
H₂N—CH₂—SO₃R (1)
in which R is an alkyl group having 1 to 6 carbon(s).

4. The medicinal composition according to claim 1, wherein said composition is in a form suitable for inhibition of production of TNFα.

5. The medicinal composition according to claim 1, wherein said composition is in a form suitable for treatment of liver diseases.

6. The medicinal composition according to claim 1, wherein the aminomethanesulfonic acid is in a form of a salt.

7. The medicinal composition according to claim 6, wherein the salt is selected from the group consisting of sodium salt, potassium salt, calcium salt, magnesium salt and ammonium salt.

8. The medicinal composition according to claim 1, wherein said composition is in a form selected from the group consisting of a solution, a granule, a tablet, and a powder.

9. The medicinal composition according to claim 8, wherein said aminomethanesulfonic acid is present at a concentration suitable to provide 1 mg to 10 g per day to a patient in need thereof.

10. The medicinal composition according to claim 1, further comprising one or more additives selected from the group consisting of fillers, diluents, additives, disintegrating agents, binders, coating agents, lubricants, gliding agents, lubricating agents, flavoring agents, sweeteners, solubilizing agents, spices, dyes, and nutrients.

11. A method for inhibiting the production of TNFα, comprising administering to a patient in need thereof an effective amount of a medicinal composition comprising aminomethanesulfonic acid.

12. The method according to claim 11, wherein the aminomethanesulfonic acid is in a form of a salt or an ester.

13. The method according to claim 11, wherein said effective amount is 1 mg to 10 g per day.

14. The method according to claim 11, wherein said effective amount is 10 mg to 5 g per day per day.

15. The method according to claim 11, wherein said effective amount is 100 mg to 1 g per day.

16. A method for treatment, amelioration and/or prevention of liver diseases, comprising administering to a patient in need thereof an effective amount of a medicinal composition comprising aminomethanesulfonic acid.

17. The method according to claim 16, wherein the aminomethanesulfonic acid is in a form of a salt or an ester.

18. The method according to claim 16, wherein said effective amount is 1 mg to 10 g per day.

19. The method according to claim 16, wherein said effective amount is 10 mg to 5 g per day per day.

20. The method according to claim 16, wherein said effective amount is 100 mg to 1 g per day.
